(19) United States
(12) Patent Application Publication
(10) Pub. No.: US 2018/0194794 A1
(43) Pub. Date: Jul. 12, 2018

(54) Activated N-Acetylated Sugars and Oligosaccharides

(71) Applicant: zuChem, Inc., chicago, IL (US)

(72) Inventor: Leila Aminova, East Peoria, IL (US)

(21) Appl. No.: 15/736,126

(22) PCT Filed: Jun. 15, 2016
(86) PCT No.: PCT/US16/37577

§ 371 Date: Dec. 13, 2017

Related U.S. Application Data
(60) Provisional application No. 62/175,699, filed on Jun. 15, 2015.

(51) Int. Cl. C07H 19/10; C07H 1/08; C07H 3/06; C12P 19/18; C12N 9/10; C12N 9/12; C12N 9/90

(52) U.S. Cl. CPC C07H 19/10; C07H 1/08; C07H 3/06; C12Y 207/07012; C12N 9/1048; C12N 9/1241; C12N 9/90; C12P 19/18

(57) ABSTRACT

The invention relates to production of uridine-5′diphospho-N-acetylglucosamine and uridine-5′diphospho-N-acetylgalactosamine. The invention further relates to the production of lacto-/V-triose II and globotetraose.

### PRIORITY

This application claims the benefit of U.S. Ser. No. 62/175,699, filed on Jun. 15, 2015, which is incorporated by reference in its entirety herein.

### GOVERNMENT INTERESTS

This invention was made with government support under NIH Grant Number 1R43GM110916. The U.S. Government has certain rights in this invention.

### BACKGROUND OF THE INVENTION

Uridine-5′-diphospho-N-acetylglucosamine (UDP-GlcNAc) and uridine-5′-diphospho-N-acetylgalactosamine (UDP-GalNAc) participate in many biological processes. They comprise the core structures of glycans in glycoproteins (Bioorg Med Chem, 2005. 13(17): p. 5021-34; Biochimie, 1988. 70(11): p. 1521-33) and glycolipids. They are important components of human milk oligosaccharides and blood antigenic determinants. Presently, oligosaccharides with core structures containing these sugars are needed for investigating cell signaling processes and metabolic regulation; these oligosaccharides have intensively been investigated as antimicrobial agents and prospective anticancer vaccines (Immunol Cell Biol, 2005. 83(4): p. 418-28; Glycobiology, 1993. 3(2): p. 97-130; Vaccine, 2011. 29(48): p. 8802-26; Annu Rev Nutr, 2005. 25: p. 37-58; J Nutr, 2005. 135(5): p. 1308-12; J Nutr, 2005. 135(5): p. 1304-7; Org Biomol Chem, 2011. 9(10): p. 3598-610).

### SUMMARY OF THE INVENTION

The invention provides (1) methods of synthesizing uridine-5′-diphospho-N-acetylglucosamine (UDP-GlcNAc) using chitin as a starting material. The methods comprise contacting chitin with an exochitanase to produce N,N-diacetylchitobiose (chitobiose). The methods comprise contacting N,N-diacetylchitobiose with a N,N-diacetylchitobiose phosphorylase, in the presence of inorganic phosphate, to produce N-acetylglucosamine (GlcNAc) and/or GlcNAc-1-phosphate. The methods comprise contacting the N-acetylglucosamine (GlcNAc) with a N-acetylhexosamine kinase, in the presence of ATP, to produce GlcNAc-1-phosphate. The methods comprise contacting GlcNAc-1-phosphate with a uridylyltransferase, in the presence of UTP, to produce UDP-GlcNAc. In an embodiment, the methods may be practiced in individual steps with additions of reaction components. In another embodiment, the methods may be practiced in a single reaction vessel with all reaction components present at the start of the reactions.

The invention provides (2) methods of synthesizing uridine-5′diphospho-N-acetylgalactosamine (UDP-GalNAc) using chitin as a starting material. The methods comprise contacting chitin with an exochitanase to produce N,N-diacetylchitobiose. The methods comprise contacting N,N-diacetylchitobiose with a N,N-diacetylchitobiose phosphorylase, in the presence of inorganic phosphate, to produce N-acetylglucosamine (GlcNAc) and/or GlcNAc-1-phosphate. The methods comprise contacting the N-acetylglucosamine (GlcNAc) with a N-acetylhexosamine kinase, in the presence of ATP, to produce GlcNAc-1-phosphate. The methods comprise contacting GlcNAc-1-phosphate with a uridylyltransferase, in the presence of UTP, to produce UDP-GlcNAc. The methods comprise contacting UDP-GlcNAc with a UDP-glucose-4-epimerase to produce UDP-GalNAc. In an embodiment, the methods may be practiced in individual steps with additions of reaction components. In another embodiment, the methods may be practiced in a single reaction vessel with all reaction components present at the start of the reactions. In yet another embodiment, the UDP-glucose-4-epimerase is a recombinant epimerase originally isolated from Vulcanisaeta moutnovskia (GalE-VM) (SEQ ID NO:2) or a recombinant epimerase originally isolated from Thermus thermophilus JL18 (GalE-TT) (SEQ ID NO:4) or a recombinant epimerase originally isolated from Pyrobaculum calidifontis (GalE-PC) (SEQ ID NO:6).

The invention provides (3) methods of synthesizing uridine-5′diphospho-N-acetylglucosamine (UDP-GlcNAc) using N,N-diacetylchitobiose as a starting material. The methods comprise contacting N,N-diacetylchitobiose with a N,N-diacetylchitobiose phosphorylase, in the presence of inorganic phosphate, to produce N-acetylglucosamine (GlcNAc) and/or GlcNAc-1-phosphate. The methods comprise contacting the N-acetylglucosamine (GlcNAc) with a N-acetylhexosamine kinase, in the presence of ATP, to produce GlcNAc-1-phosphate. The methods comprise contacting GlcNAc-1-phosphate with a uridylyltransferase, in the presence of UTP, to produce UDP-GlcNAc. In an embodiment, the methods may be practiced in individual steps with additions of reaction components. In another embodiment, the methods may be practiced in a single reaction vessel with all reaction components present at the start of the reactions.

The invention provides (4) methods of synthesizing uridine-5′diphospho-N-acetylgalactosamine (UDP-GalNAc) using N,N-diacetylchitobiose as a starting material. The methods comprise contacting N,N-diacetylchitobiose with a N,N-diacetylchitobiose phosphorylase, in the presence of inorganic phosphate, to produce N-acetylglucosamine (GlcNAc) and/or GlcNAc-1-phosphate. The methods comprise contacting the N-acetylglucosamine (GlcNAc) with a N-acetylhexosamine kinase, in the presence of ATP, to produce GlcNAc-1-phosphate. The methods comprise contacting GlcNAc-1-phosphate with a uridylyltransferase, in the presence of UTP, to produce UDP-GlcNAc. The methods comprise contacting UDP-GlcNAc with a UDP-glucose-4-epimerase to produce UDP-GalNAc. In an embodiment, the methods may be practiced in individual steps with additions of reaction components. In another embodiment, the methods may be practiced in a single reaction vessel with all reaction components present at the start of the reactions. In yet another embodiment, the UDP-glucose-4-epimerase is a recombinant epimerase originally isolated from Vulcanisaeta moutnovskia (GalE-VM) (SEQ ID NO:2) or a recombinant epimerase originally isolated from Thermus thermophilus JL18 (GalE-TT) (SEQ ID NO:4) or a recombinant epimerase originally isolated from Pyrobaculum calidifontis (GalE-PC) (SEQ ID NO:6).

The invention provides (5) methods of synthesizing uridine-5′diphospho-N-acetylglucosamine (UDP-GlcNAc) using N-acetylglucosamine as a starting material. The methods comprise contacting the N-acetylglucosamine (GlcNAc) with a N-acetylhexosamine kinase, in the presence of ATP, to produce GlcNAc-1-phosphate. The methods comprise contacting GlcNAc-1-phosphate with a uridylyltransferase, in the presence of UTP, to produce UDP-GlcNAc. In an embodiment, the methods may be practiced in individual steps with additions of reaction components. In another embodiment, the methods may be practiced in a single reaction vessel with all reaction components present at the start of the reactions.

The invention provides (6) methods of synthesizing uridine-5′diphospho-N-acetylgalactosamine (UDP-GalNAc) using N-acetylglucosamine as a starting material. The methods comprise contacting the N-acetylglucosamine (GlcNAc) with a N-acetylhexosamine kinase, in the presence of ATP, to produce GlcNAc-1-phosphate. The methods comprise contacting GlcNAc-1-phosphate with a uridylyltransferase, in the presence of UTP, to produce UDP-GlcNAc. The methods comprise contacting UDP-GlcNAc with a UDP-glucose-4-epimerase to produce UDP-GalNAc. In an embodiment, the methods may be practiced in individual steps with additions of reaction components. In another embodiment, the methods may be practiced in a single reaction vessel with all reaction components present at the start of the reactions. In yet another embodiment, the UDP-glucose-4-epimerase is a recombinant epimerase originally isolated from Vulcanisaeta moutnovskia (GalE-VM) (SEQ ID NO:2) or a recombinant epimerase originally isolated from Thermus thermophilus JL18 (GalE-TT) (SEQ ID NO:4) or a recombinant epimerase originally isolated from Pyrobaculum calidifontis (GalE-PC) (SEQ ID NO:6).

The invention provides (7) methods of synthesizing lacto-N-triose II (LNT II), comprising: contacting lactose and UDP-GlcNAc with a glycosyltransferase to produce lacto-N-triose II.

The invention provides (8) methods of synthesizing globotetraose, comprising: contacting globotriose and UDP-GlcNAc with a glycosyltransferase to produce globotetraose.

### BRIEF DESCRIPTION OF THE DRAWINGS

FIG. 1: UDP-GlcNAc and UDP-GalNAc production from chitin, or N,N-diacetylchitobiose, or GlcNAc, or GlcNAc-1-phosphate.

FIG. 2: Expression of ChiB from P. furiosus on E. coli BI21: 1. LB, 2. LB with 5 mg/ml casaminoacids, 3. LB with 10 mg/ml casaminoacids, 4. LB with 10 mg/ml casaminoacids and 1% glucose.

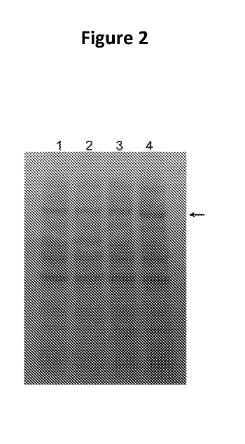

FIG. 3: HPLC analysis of chitinase reaction after 16 h incubation at 95° C. for P. furiosus chitinase and at 55° C. for B. cereus chitinase with crystal or colloidal.

FIG. 4: Chitobiose (N,N′-diacetylchitobiose) purification. Arrow indicates chitobiose.

FIG. 5: N,N′-diacetylchitobiose phosphorylase (CHP-P) and N-acetylhexosamine kinase (HK) reactions after 72 h incubation. 1. 20 mM GlcNAc-1-phosphate, standard, 2. CHB-P only with chitobiose, 3. HK with chitobiose, 4. CHB-P and HK with chitobiose, 4. HK with GlcNAc, incubation at 30° C., 5. HK with GlcNAc, incubation at 37° C.

FIG. 6: HPLC analysis (Aminex® HPX-87 column) of reactions with CHB-P and HK. CHB-P—reaction only with CHB-P, CHB-P&HK-coupled reaction with CHB-P and HK. 1—Chitobiose, 2—GlcNAc.

FIG. 7: Nucleotidyltransferase reaction with purified GlcNAc-1-phosphate and Streptoccocus thermophilus uridylyltransferase. Reaction was monitored by TLC (left) and HPLC (Inertsil ODS-4).

FIG. 8: GlcNac (1) and GalNac (2) after acid degradation of epimerase reaction on Amenex® HPX-87 column.

FIG. 9: Commercial UDP-GlcNAc (1), UDP-GalNAc (2) and mix appearance after running on Inertsil ODS-4 column.

FIG. 10: Glycosyltransferase reaction with LgtA enzyme and commercial UDP-GlcNAc (R1), mix of commercially available UDP-GlcNAc and UDP-GalNAc (R2) and mix produced by epimerase reaction (R3). A—lacto-N-triose II peak formation analyzed by LC-NH2 column (Supelcosil) and B—activated sugar consumption during reaction by Inertsil ODS-4 column.

FIG. 11: Initial Epimerase Analysis. One or two clones from each plasmid transformation (numbered 1 or 2) were analyzed for expression and presence of soluble proteins. Abbreviations are described in Table 1. Lanes include: TT GalE from Thermus thermophilus, EC GalE from Escherichia coli J53 mutant); VM GalE from Vulcanisaeta moutnovskia; PC GalE from Pyrobaculum calidifontis.

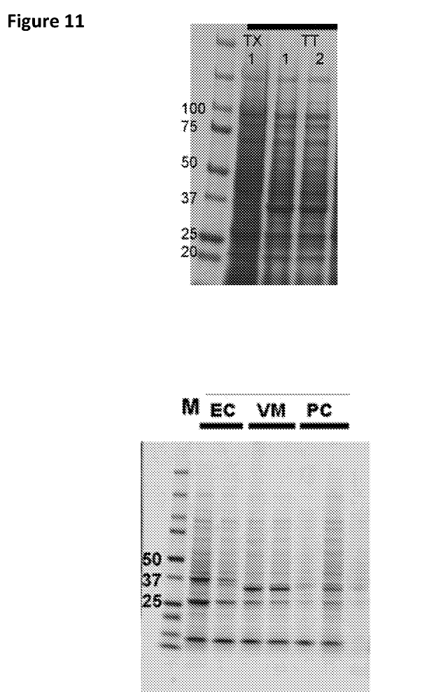

FIG. 12: Coupling of UDP-GlcNAc/GalNAc production reaction with a glycotransferase to produce lacto-N-triose II and globotetraose.

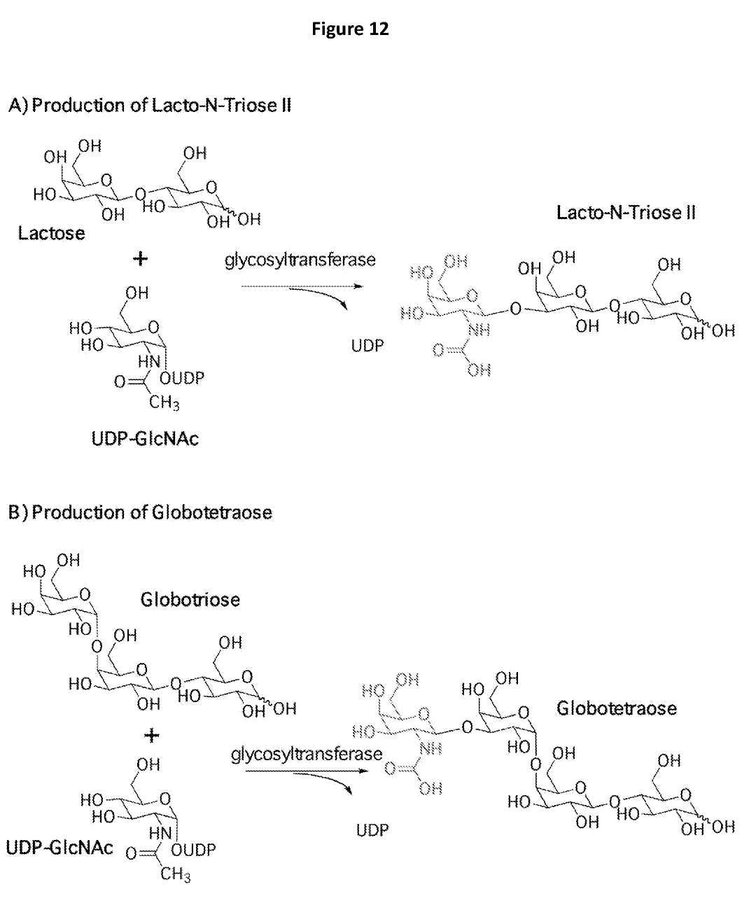

FIG. 13: Nucleic Acid (SEQ ID NO:1) and Amino Acid (SEQ ID NO:2) Sequence for epimerase cloned from Vulcanisaeta moutnovskia (GalE-VM).

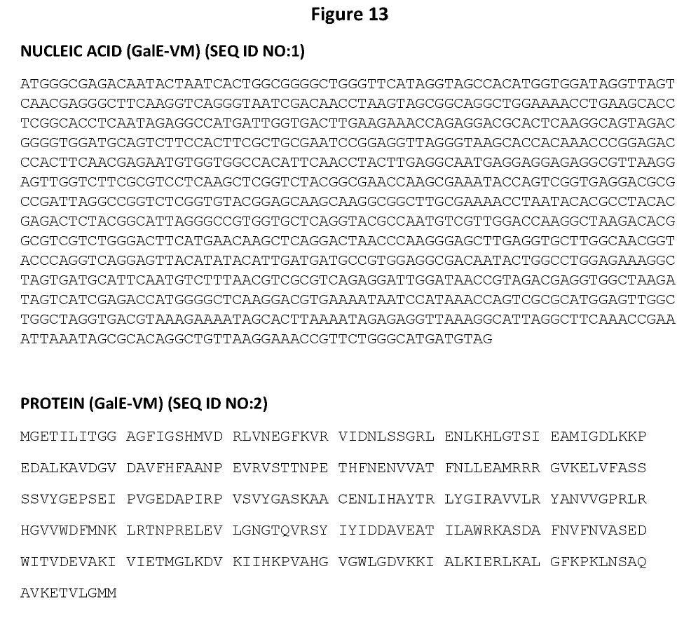

FIG. 14: Nucleic Acid (SEQ ID NO:3) and Amino Acid (SEQ ID NO:4) Sequence for epimerase cloned from Thermus thermophilus (GalE-TT).

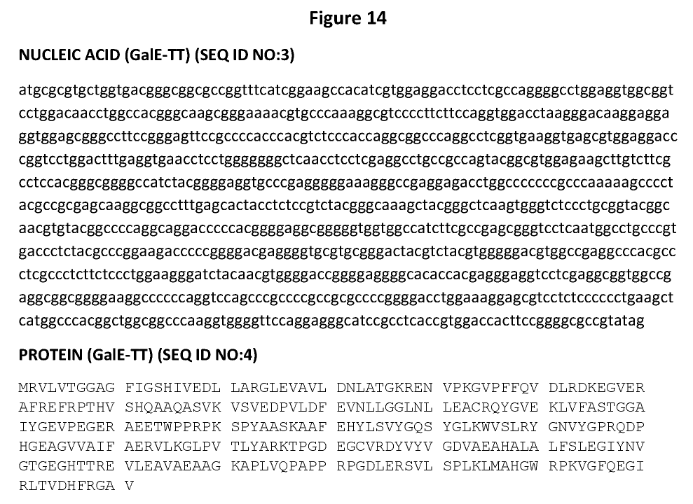

FIG. 15: Nucleic Acid (SEQ ID NO:5) and Amino Acid (SEQ ID NO:6) Sequence for epimerase cloned from Pyrobaculum calidifontis (GalE-PC).

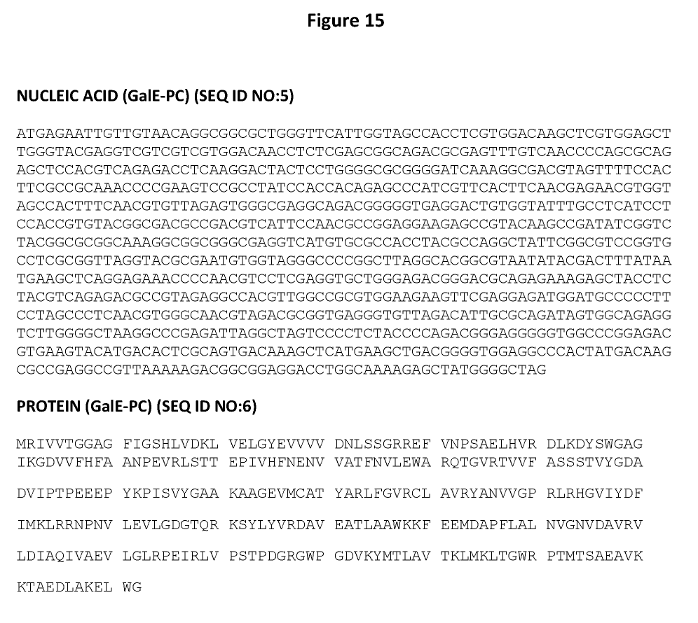

FIG. 16: Recombinant production of epimerases from Pyrobaculum calidifontis (PC), Thermus thermophilus (TT), Vulcanisaeta moutnovskia (VM), in E. coli (EC).

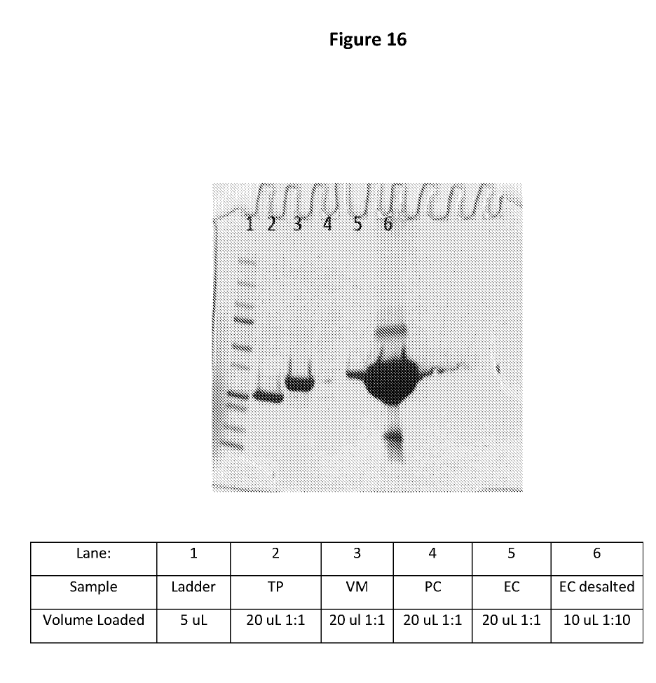

### DETAILED DESCRIPTION OF THE INVENTION

As used herein, the singular forms “a,” “an,” and “the” include plural referents unless the context clearly dictates otherwise. The term “about” in association with a numerical value means that the numerical value can vary plus or minus by 5% or less of the numerical value. The term “synthesize” or “synthesizing” or “produce” or “producing” is used interchangeably herein.

The terms “polynucleotide”, “polynucleotide sequence”, “nucleic acid” and “nucleic acid sequence” are used interchangeably herein. These terms encompass nucleotide sequences and the like. A polynucleotide may be a polymer of DNA or RNA that is single- or double-stranded, that optionally contains synthetic, non-natural or altered nucleotide bases. A polynucleotide may be comprised of one or more segments of cDNA, genomic DNA, synthetic DNA, or mixtures thereof.

The terms “protein”, “enzyme”, “amino acid”, and “amino acid sequence” are used interchangeably herein. These terms encompass isolated native and isolated non-native forms, native and recombinant forms of proteins wherein the isolated or recombinant forms may differ from that found in nature. The chemical nature of the protein may possess three dimensional folding different than that found in nature; it may be chemically modified with thiol-containing reagents, for example, which aid in the stability and storage of the compound; it may be stored in medium which enables its long-term stability in isolation (such as with glycerol).

The term “gene” as used herein refers to a polynucleotide sequence that expresses a protein, and which may refer to the coding region alone or may include regulatory sequences upstream and/or downstream to the coding region (e.g., 5′ untranslated regions upstream of the transcription start site of the coding region). A gene that is “native” or “endogenous” refers to a gene as found in nature with its own regulatory sequences; this gene is located in its natural location in the genome of an organism. “Chimeric gene” refers to any gene that is not a native gene, comprising regulatory and coding sequences that are not found together in nature. A “foreign” or “heterologous” gene refers to a gene that is introduced into the host organism by gene transfer. Foreign genes can comprise native genes inserted into a non-native organism, native genes introduced into a new location within the native host, or chimeric genes. The polynucleotide sequences in certain embodiments disclosed herein are heterologous. A “transgene” is a gene that has been introduced into the genome by a transformation procedure. A “codon-optimized gene” is a gene having its frequency of codon usage designed to mimic the frequency of preferred codon usage of the host cell.

A native amino acid sequence or polynucleotide sequence is naturally occurring, whereas a non-native amino acid sequence or polynucleotide sequence does not occur in nature.

“Coding sequence” as used herein refers to a DNA sequence that codes for a specific amino acid sequence. “Regulatory sequences” as used herein refer to nucleotide sequences located upstream of the coding sequence's transcription start site, 5′ untranslated regions and 3′ non-coding regions, and which may influence the transcription, RNA processing or stability, or translation of the associated coding sequence. Regulatory sequences may include promoters, enhancers, silencers, 5′ untranslated leader sequence, introns, polyadenylation recognition sequences, RNA processing sites, effector binding sites, stem-loop structures and other elements involved in regulation of gene expression.

The term “recombinant” as used herein refers to an artificial combination of two otherwise separated segments of sequence, e.g., by chemical synthesis or by the manipulation of isolated segments of nucleic acids by genetic engineering techniques. The terms “recombinant”, “transgenic”, “transformed”, “engineered” or “modified for exogenous gene expression” are used interchangeably herein.

The term “transformation” as used in certain embodiments refers to the transfer of a nucleic acid molecule into a host organism. The nucleic acid molecule may be a plasmid that replicates autonomously, or it may integrate into the genome of the host organism. Host organisms containing the transformed nucleic acid fragments are referred to as “transgenic” or “recombinant” or “transformed” organisms or “transformants”.

The term “recombinant” or “heterologous” refers to an artificial combination of two otherwise separated segments of sequence, e.g., by chemical synthesis or by the manipulation of isolated segments of nucleic acids by genetic engineering techniques.

Any enzyme, used in the process of the invention as disclosed herein, may be sourced from eukaryotic or prokaryotic sources, both naturally occurring and recombinant forms.

The term “chitobiose” may be used interchangeably with “N,N′-diacetylchitobiose”.

The temperature of the reaction solution in which a substrate and enzyme are contacted can be controlled, if desired. In certain embodiments, the solution has a temperature between about 25° C. to about 100° C. The temperature of the solution in certain other embodiments is between about 30° C. to about 95° C. Alternatively, the temperature of the solution may be about 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94° C.

The temperature of the reaction solution may be maintained using various means known in the art. For example, the temperature of reaction solution can be maintained by placing the vessel containing the reaction solution in an air or water bath incubator set at the desired temperature.

The pH of the reaction solution in which a substrate and enzyme are contacted can be between about 4.0 to about 8.0 in certain embodiments. Alternatively, the pH can be about 4.0, 4.5, 5.0, 5.5, 6.0, 6.5, 7.0, 7.5, or 8.0. In certain embodiments, the pH of a solution containing the substrate may be set before adding the particular enzyme. The pH of the reaction solution can be adjusted or controlled by the addition or incorporation of a suitable buffer, including but not limited to: phosphate, Tris, citrate, or a combination thereof. The concentration of the buffer can be from 0 mM to about 100 mM, for example. In certain embodiments, the buffer concentration is about 10, 20, or 50 mM.

The purity of end products, such as N,N diacetylchitobiose, GlcNAc, GlcNAc-1-phosphate, UDP-GlcNAc, UDP-GalNAc, lacto-N-triose II, and globotriose, in certain embodiments is about 40% to 99%. Alternatively, purity of end products can be about 50% to 59%, or about 60% to 69% or about 70% to 79%, or about 80% to 89% or about 90% to 95%. In other embodiments, purity of end products is about 99%.

The yield of UDP-GlcNAc, UDP-GalNAc, lacto-N-triose II or globotriose is about 1 g to 10 kg. In certain embodiments, yield of UDP-GlcNAc, UDP-GalNAc, lacto-N-triose II or globotriose is about 2 g to 5 g, or about 6 g to 24 g, or about 25 g to 100 g, or about 101 g to 500 g, or about 501 g to 999 g, or about 1 kg to 5 kg or about 6 kg to 10 kg.

The particular substrate and an enzyme of the respective reaction step are contacted in a reaction solution. It will be understood that, as the particular enzyme synthesizes a reaction product, the reaction solution becomes a reaction mixture. The contacting step of the disclosed process can be performed in any number of ways. For example, the desired amounts of substrate and enzyme(s) may be added sequentially or at one time (optionally, other components may also be added at this stage, such as buffer components). The solution may be kept still, or agitated via stirring or orbital shaking, for example. The reaction(s) of the present invention can be cell-free, e.g. in vitro, or including cells, e.g. in vivo.

Surprisingly, it has been found that when more than one enzyme is present simultaneously, yield improves. In a non-limiting example, when N,N-diacetylchitobiose is incubated with N,N-diacetylchitobiose phosphorylase alone (100 mM Sodium phosphate buffer, pH 7.5, 85 mM N,N-diacetylchitobiose, 10 mM MgSO4, 0.4 mg N,N-diacetylchitobiose phosphorylase) product yield was 20%. When GlcNAc is incubated with N-acetylhexosamine kinase alone (100 mM Sodium phosphate buffer, pH 7.5, 40 mM GlcNAc, 10 mM MgSO4, 40 mM ATP, 0.7 mg of N-acetylhexosamine kinase) product yield was about 10%. In stark comparison, and as an unexpected result of co-incubation, when N,N-diacetylchitobiose is co-incubated with N,N-diacetylchitobiose phosphorylase and N-acetylhexosamine kinase (100 mM Sodum phosphate buffer, pH 7.5, 85 mM chitobiose, 10 mM MgSO4, 40 mM ATP, 0.4 mg N,N-diacetylchitobiose phosphorylase and 0.7 mg of N-acetylhexosamine kinase) product yield was about 90-95%. Thus, simultaneous addition of reaction enzymes in the process of this invention, may in some embodiments produce greater yield.

The enzyme can be added to the reaction where the solution at the end of reaction preparation, which includes buffer, salt and substrate at the desired pH. This approach can avoid enzyme precipitation. The pH of such a preparation can then be modified as desired. The reaction can be carried out to completion without any added buffer, if desired.

Completion of the reaction in certain embodiments can be determined HPLC or thin layer chromatography. Typically, a reaction of the disclosed process will take about 12, 24, 36, 48, 60, 72, 84, or 96 hours to complete, depending on certain parameters such as the amount of substrate and enzyme are used in the reaction.

Recent developments in chemoenzymatic synthesis of oligosaccharides using recombinant glycosyltransferases provide a powerful tool for obtaining biologically important glycans. The cost of production of activated sugars for many applications, however, remains high due to a number of factors. These include the lack of effective enzymatic systems for making these activated sugars, extensive purification steps, and the cost of nucleotide triphosphates. This has created a need to find less expensive solutions to produce these activated sugars. The present invention results in more efficient production and purification of end products.

A. Importance of UDP-GlcNAc and UDP-GalNAc

UDP-GlcNAc and UDP-GalNAc are the key building blocks for human milk oligosaccharides, blood antigens, and other important oligosaccharides. Table 1 describes some important natural glycans with GlcNAc or GalNAc in core structures.

TABLE 1
Natural glycans with GlcNAc or GalNAc in core structures

| Contains GlcNAc | Contains GalNAc |
|---|---|
| Lewis blood group antigens | Globotetraose and derivatives |
| Globotriose and derivatives | GloboH |
| Lacto-N-tetraose (LNT) | Forssman antigen |
| and derivatives | |
| Lacto-N-neo-tetraose (LNnT) | Mucin type O-linked glycoproteins |
| and derivatives | |

Glycoproteins and Glycolipids contain many oligosaccharides and polysaccharides that incorporate UDP-GlcNAc and UDP-GalNAc. Oligosaccharides that are attached to proteins and lipids play an important role in many cellular processes and can act as effective modulators of these processes in eukaryotes and, therefore, have tremendous potential as pharmaceuticals.

Human Milk Oligosaccharides (HMOs).

HMOs have been shown to have a prebiotic effect by modulating bacterial-host interactions and intestinal microbiota composition as well as by reducing the possibility of infections due to interference with the adhesion of pathogenic bacteria (Nutr Rev, 2009. 67 Suppl 2: p. S183-91; Br J Nutr, 2007. 98 Suppl 1: p. S74-9). Over 200 different HMOs have been identified, with up to 70% of them containing LNT, LNnT and their derivatives (J Agric Food Chem, 2006. 54(20): p. 7471-80; Proc Natl Acad Sci USA, 2011. 108 Suppl 1: p. 4653-8). Both of these contain a lactose-reducing end elongated with GlcNAc. The LNnT derivative lacto-N-fucopentaose III (LNFPIII) is found in schistosome eggs and in breast milk and is able to suppress host immune responses, has therapeutic efficacy in mouse models of psoriasis and type 1 diabetes (Immunol Rev, 2009. 230(1): p. 247-57), and prolongs heart transplant survival (Dutta, P., et al., Lacto-N-fucopentaose III, a Pentasaccharide, Prolongs Heart Transplant Survival. Transplantation, 2010) [15]. Research with helminthes (parasitic worms) also containing LNFPIII on its surface have demonstrated a great potential in down-regulation of inflammatory responses both in vitro and in vivo, in various disease models, and, in some cases, in clinical trials (IUBMB Life, 2010. 62(4): p. 303-12; Adv Exp Med Biol, 2009. 666: p. 157-66. Treatment of humans with live parasitic worms obviously is impractical and imposes a risk of infection and unexpected side effects. Purified glycans would be a better and safer alternative if they could be made in sufficient quantities.

While the importance of HMOs in infant health and development of immune response is generally agreed to, the full potential of these sugars cannot be investigated due to lack of pure oligosaccharides even for pre-clinical studies. The small amounts of sugars that can be purified from donors milk still contain lipopolysaccharides and other components that make interpreting results difficult. Developing technology now for large scale production of key building blocks of HMO and other important oligosaccharides will help researchers find new methods to treat and cure diseases as well as affordably produce them as food additives.

Anti-Infective Agents.

The rise of antibiotic-resistant pathogens poses a major problem for healthcare globally. An attractive alternative to antibiotics is the anti-infective activity of oligosaccharides, including human milk oligosaccharides, which act through: (i) anti-adhesive activity, by blocking receptors for toxins and bacteria; (ii) prebiotic activity that promotes the growth of beneficial bacteria; and (iii) immunomodulatory activities (Annu Rev Nutr, 2005. 25: p. 37-58; J Nutr, 2005. 135(5): p. 1308-12; J Nutr, 2005. 135(5): p. 1304-7; Proc Natl Acad Sci USA, 2011. 108 Suppl 1: p. 4653-8; Bacterial and Viral Infections. 2009; Biological Roles of Glycans. 2009; Glycobiology, 2008. 18(10): p. 747-9).

The GalNAc containing oligosaccharides globotetraose and isoglobotetraose also play significant roles as receptors, especially in the adhesion of human embryonic carcinoma cells, pathogenesis of urinary tract infections, and human parvovirus B19 infection (Science, 1993. 262(5130): p. 114-7; Mol Diagn, 2001. 6(4): p. 307-12; Annu Rev Biochem, 1989. 58: p. 309-50; Infect Immun, 1998. 66(8): p. 3856-61). Paragloboside, attached to ceramide, has been isolated from human erythrocytes and polymorphonuclear leukocytes, and from some naturally occurring antibodies (J Immunol, 1977. 118(4): p. 1415-9). Oligosaccharide production methods are needed for carbohydrate-based vaccines, glycomimetic drugs, carbohydrate-based diagnostics, and prebiotics. In addition, current extensive research on the role of oligosaccharides in various cellular processes requires the custom production of oligosaccharides at reasonable costs.

In therapy, oligosaccharide receptor mimetics have been shown to reduce infection in vivo. The symptoms of pneumococcal pneumonia were alleviated in a rabbit model by administration of the HMO LNnT and its α2-3- or α2-6-sialylated derivatives (J Infect Dis, 1997. 176(3): p. 704-12). In BALB/c mice mannose and globotetraose inhibited urinary tract infections caused by E. coli (Nature, 1982. 298(5874): p. 560-2).

Vaccines for Cancer Treatment.

Aberrant glycosylation is associated with many tumors and usually correlates with poor clinical prognosis (Nat Rev Drug Discov, 2005. 4(6): p. 477-488; Cancer Science, 2004. 95(5): p. 377-384). A number of tumor associated carbohydrate antigens contain GalNAc and/or GlcNAc including (but not limited to) the mucin related (0-linked) Tn antigen, Thomsen-Friedenreich antigen (TF), the blood group Lewis related Lewis^{x}, Sialyl Lewis^{x}, Sialyl Lewis^{a}, the glycosphingolipids GloboH, the and gangliosides GM2, GD2, GD3, fucosyl GM1 (Vaccine, 2011. 29(48): p. 8802-26; Cellular Organization of Glycosylation. 2009; Immunol Cell Biol, 2005. 83(4): p. 429-39; Expert Rev Vaccines, 2009. 8(10): p. 1399-413; Curr Opin Chem Biol, 2009. 13(5-6): p. 608-17).

Cancer.

An increased understanding of the key roles carbohydrates play in metastasis has resulted in significant effort being applied to develop effective vaccines. Synthesizing antigens to mimic the cancer-specific cell surface antigen and induce an immune response could lead to novel cancer treatments. Carbohydrate-based vaccines mimic the tumor cell surface. Vaccines against tumor associated mucin antigens (STn, TF and Tn) displayed in a clustered format developed robust immune responses (Cancer Immunol Immunother, 1999. 48(1): p. 1-8). The most recent vaccines incorporated several antigens (multivalent vaccine). A vaccine containing three different antigens (GloboH, Lewis^{y} and Tn) has been evaluated in a murine model (Proc Natl Acad Sci USA, 2002. 99(21): p. 13699-704). A clinical trial with the synthetic version of fucosyl-GM1 was performed in patients with lung cancer (Clin Cancer Res, 2004. 10(18 Pt 1): p. 6094-100). Five out of six patients generated an IgM response. A fully synthetic GloboH was studied in a patient with prostate cancer and showed a positive effect (Proc Natl Acad Sci USA, 1999. 96(10): p. 5710-5). Antibody response to treatment with GloboH was detected in breast cancer patients (Proc Natl Acad Sci USA, 2001. 98(6): p. 3270-5). Other vaccines based on carbohydrates are in developmental stages (Vaccine, 2011. 29(48): p. 8802-26; J Med Chem, 2012. 55(15): p. 6968-74; Cancer Sci, 2013. 104(3): p. 282-90; Cancer Immunol Immunother, 2009. 58(2): p. 187-200). Monovalent vaccine candidates have also shown immune response (Chembiochem, 2009. 10(3): p. 455-63; Cancer Immunol Immunother, 2009. 58(9): p. 1397-405).

Need for Larger Volumes of these Building Blocks.

Thus, various oligosaccharides all of which require UDP-GlcNAc or UDP-GalNAc as building blocks are needed on a large scale as core structure for therapeutics, drug discovery as well as nutritional supply and in food. The development of technology for production of these activated sugars at large scale will promote further development in drug discovery and improve human health.

Current technologies, however, are able to provide only milligram quantities of oligosaccharides while preclinical/clinical research require gram quantities for safety assessment, dose-ranging, and efficacy trials in cohorts at high risk of gastrointestinal infections. A promising approach to solve these issues is the in vitro or in vivo use of glycosyltransferases with simple starting materials such as monosaccharides or readily available polysaccharides and nucleotide-activated sugars. The implementation of cost-effective production of oligosaccharide building blocks will enable their use not only in drug discovery and production but also as prebiotic food supplements or in infant formula.

B. Current Methods of Production of Activated N-Acetylated Sugars

A number of methods have been explored to produce the activated N-acetylated sugar building blocks including the following:

1. UDP-GlcNAc can be purified from baker's yeast. Only low levels of activated sugars are present in the cytosol and this results in expensive purification steps (J Biol Chem, 1953. 203(2): p. 1055-70).

2. Methods that use metabolically engineered E. coli and Corynebacterium ammoniagenes cells and orotic acid and glucosamine as starting materials may accumulate 7.8 g/L after 8 h incubation (Biotechnology Letters, 2000. 22(6): p. 479-483). Another group modified Lactobaccillus casei for the production of UDP-GlcNAc by overexpressing the corresponding biosynthetic genes but reported only a 1.5-fold increase in the intracellular UDP-GlcNAc level (Bioengineered, 2012. 3(6): p. 339-42). This method still needs further improvement to achieve more significant production rates. The use of metabolically engineered bacteria is a very promising approach for activated sugar production, because it does not require expensive starting materials, but the final product is accumulated in the cytoplasm, which is limiting. The use of the permeabilised cells results in complicated purification steps (because all small molecules will be released to media) that increase the cost of the final product. In our experience, for example, product purification can require more time and effort than the production itself, because it is more difficult to separate desired activated sugars from the pool of other endogenous activated sugars. This type of engineering can be useful if the desired product is secreted into the growth media.

3. Presently, chemoenzymatic synthesis has become the method of choice for the synthesis of various activated sugars and oligosaccharides. Several sugar-1-phosphate kinases have been characterized and modified for broader substrate specificity. However, only two N-acetylhexosamine-1-kinases have been characterized (Appl Environ Microbiol, 2007. 73(20): p. 6444-9; Bioorg Med Chem Lett, 2013. 23(15): p. 4303-7) and chimeric enzymes constructed (Biotechnol Lett, 2012. 34(7): p. 1321-6). In general, the formation of UDP-GlcNAc in nature occurs via acetylation of Glucosamine-6-phosphate using acetyl CoA as a donor, after mutase transformation of N-acetylglucosamine-6-phosphate to GlcNAc-1-phospate. The last reaction is reversible and produces a mixture of GlcNAc-1-phosphate and GlcNAc-6-phospate. This is converted to GlcNAc-1-phosphate by the action of N-acetylglucosamine mutase. A uridylyltransfease finally activates the sugar using UTP to produce UDP-GlcNAc. Recently, two effective systems for UDP-GlcNAc and UDP-GalNAc production were demonstrated. One used two enzymes, N-acetylhexosamine-1-kinase (NahK) from Bifidobacterium longum and a uridylyltransferase from E. coli (GImU) (Nat Protoc, 2010. 5(4): p. 636-46), and the other a fusion enzyme (NahK/GImU) (Biotechnol Lett, 2012. 34(7): p. 1321-6). For UDP-GalNAc, however, the cost of the starting material, N-acetylgalactosamine remains high.

C. New Strategies for the Production of Activated N-Acetylated Sugars

Here we demonstrate a novel and non-obvious strategy for the production of important activated sugars for oligosaccharide synthesis, including UDP-GlcNAc and UDP-GalNAc, from the natural product chitin using recombinant enzymes from thermophilic bacteria. In an embodiment, N,N-diacetylchitobiose is used as a starting material to produce UDP-GlcNAc and/or UDP-GalNAc. In another embodiment, N-acetylglucosamine is used as a starting material to produce UDP-GlcNAc and/or UDP-GalNAc.

Chitin.

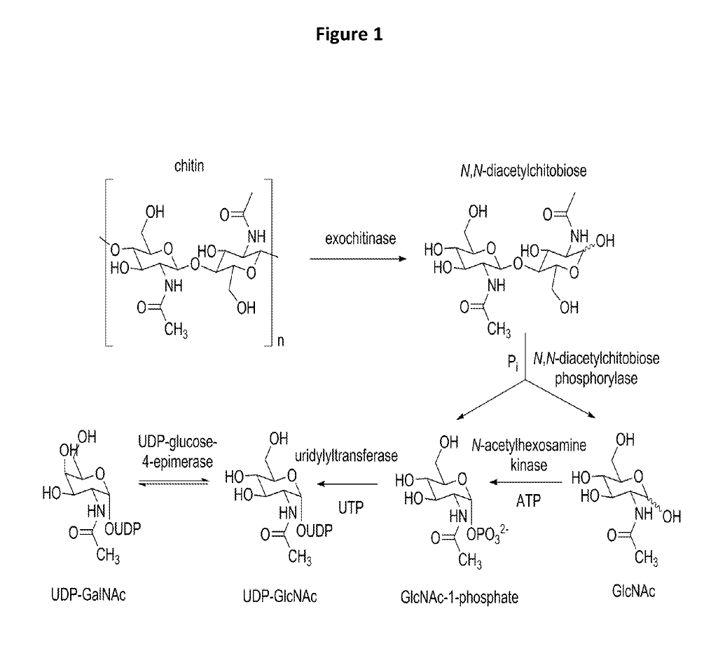

Chitin is a linear beta 1,4-linked polymer of N-acetyl-D-glucosamine (GlcNAc) (FIG. 1), and it is the second most abundant polymer in nature, with estimated yearly production of 100 billion tons. Chitin is difficult to modify chemically, but nature developed a system for the depolymerization and metabolism of chitin and chitooligosaccharides. Chitin can be hydrolyzed into oligomers and monomers by acid hydrolysis. However, enzymatic methods are more suitable allowing controllable processing and therefore predictable compounds.

Several chitinase enzymes capable of degrading chitin have been reported. Depending on the nature of enzyme, they can produce GlcNAc and N-N′-diacetylchitobiose. These enzymes originally have been investigated for the production of GlcNAc. The class of enzymes known as exochitinases, which degrades chitin mainly into N-N′-diacetylchitobiose, are useful for this process. N,N′-diacetylchitobiose phosphorylase is useful for production of GlcNAc-1-phosphate and a uridylyltransferase, for example from Streptoccocus thermophilus, will make UDP-GlcNAc. The scope of the invention disclosed herein should not be limited by the examples. Exochitanases, N,N′-diacetylchitobiose phosphorylases, and uridylyltransferases may arise from any source and be optimized for the processes disclosed herein. It is well within the ability of one skilled in the art to vary temperature, pH and reaction conditions to optimize the processes disclosed herein.

In some embodiments, the reaction steps disclosed herein may occur with sequential addition of enzyme to a reaction vessel, in some embodiments, all enzymes are present in the same reaction vessel. In still other embodiments, multiple reaction vessels may be utilized during the process. It is well within the ability of one skilled in the art to optimize reactions for the invention disclosed herein.

In some embodiments, the reactions may be carried out in an in vitro, cell-free system. In other embodiments, the reactions may be carried out in an in vivo environment wherein the reaction enzymes are provided by cell(s) producing said enzyme(s). In some embodiments, there may be multiple cells producing the reaction enzymes, in other embodiments, a single cell may be producing the reaction enzymes. In some embodiments, fermentation may be included as a step. In a specific embodiment, E. coli or yeast cells (such as Kluyveromyces lactis, Saccharomyces cerevisiae or Hansenula polymorpha) are transformed with a plasmid expressing hexosamine kinase from B. longum, nucleotydiltransferase from S. thermophilis and β1-3 glycosyltransferase (LgtA from Neisseria gonorrhoeae). Methylotrophic yeast can have some advantages because it is possible to use the temperature sensitive trehalose synthase promoter for protein induction. In yeast, the endogenous glycosyltransferase that uses UDP-GlcNAc as donor may need to be knocked out. It is well within the ability of one skilled in the art to modify and optimize the in vitro or in vivo reactions for the invention disclosed herein.

### Examples

### Material and Methods

Preparation of Colloidal Chitin.

10 grams of chitin were slowly dissolved lin 400 ml of concentrated HCl with stirring at 4° C. The mixture was incubated in a water bath at 37° C. until viscosity decreased. To this mixture 4.0 liters of sterile distilled water was added and left overnight at 4° C. The supernatant was slowly decanted and the precipitate was collected on a filter paper and washed extensively with sterile distilled water to obtain neutral pH.

Cloning and Enzyme Preparation.

The coding sequence of proteins (Table 2) were synthesized (GenScript) and cloned into pet28b expression vector. The proteins were expressed in Escherichia coli strain BL21 as His6-tagged protein and purified using cobalt resin Talon, Clontech). Protein was purified according to the manufacturer's protocol, eluted with 50 mM NaPi buffer (pH 7.8) with 300 m M NaCl and 250 mM imidazole. Fractions that contained protein were concentrated using an Amicon 10K ultrafiltration system.

To obtain chitinases, cell free extracts were heated at 60° C., centrifuged an d clear supernatant used for reaction.

TABLE 2
List of Enzymes

| Strains | Enzyme | Optimum T° | Product | Accession # |
|---|---|---|---|---|
| P. furiosus (ChiB_{cat}) | Chitinase Chi-Pf | 95° C. | N,N′- | NP_578962.1 |
| | | | diacetylchitobiose | |
| Bacillus cereus | Chitinase Chi-Bc | 60° C. | N,N′- | 3N13_A |
| | | | diacetylchitobiose | |
| Dictyoglomus | N,N′- | 50-80° C. | GlcNAc + GlcNAc- | YP_002250366.1 |
| thermophilum | diacetylchitobiose | | 1-phosphate | |
| H-6-12 | Phosphorylase | | | |
| | (CHB-P-Dt) | | | |
| Vibrio | N,N′- | 30° C. | | 1V7V_A |
| proteolyticus | diacetylchitobiose | | | |
| | Phosphorylase | | | |
| | (CHB-P-Vp) | | | |
| B. longum | N- | 37° C. | GlcNAc-1- | AB303839 |
| | acetylhexosamine | | phosphate | |
| | kinase (HK) | | | |
| Streptoccocus | Uridylyltransferase | 30° C. | UDP-GlcNAc | WP_0011226150 |
| thermophilus | | | | |

Enzyme Assays.

The following enzyme assays were used:

Chitinase activity was analyzed with powdered crystalline (Alfa Aesar) and colloidal chitin in 0.5 ml of 50 mM Tris-HCl buffer (pH 7.5) containing 10 mg of chitin and 0.3 ml of heated cell free extract. Reactions were incubated overnight at 60° C. or 90° C. Formation of chitobiose was detected by TLC or HPLC.

N,N′-diacetylchitobiose phosphorylase (CHB-P) activity was analyzed in 0.1 ml of 100 mM Sodium phosphate buffer, pH 7.5, 85 mM chitobiose, 10 mM MgSO4 and 19 ug of purified enzyme. Incubation at 30° C.

N-acetylhexosamine kinase (HK) activity was analyzed in 0.1 ml of 100 mM Sodium phosphate buffer, pH 7.5, 40 mM GlcNAc, 40 mM ATP, 10 mM MgSO4 and 35 ug of purified enzyme. Incubation at 30° C.

For combined reaction with CHB-P and HK 100 mM of Sodium phosphate buffer, pH 7.5 used, 85 mM chitobiose, 10 mM MgSO4, 40 mM ATP 19 ug of purified CHB-P and 35 ug of HK. Incubation at 30° C.

Nucleotidyltransfrease activity was analyzed in 0.1 ml of 100 mM Sodium phosphate buffer, pH 8.0, 50 mM TrisHCl, pH 8.0, 30 mM MgSO4, 30 mM GlcNAc-1-Phosphate, 30 mM UTP, 9 ug of nucleotidyltransferase. Incubation at 30° C.

Chitobiose phosphorylase reactions (200 μl). R1—reaction with Chitobiose phosphorylase was performed in 1 ml of 10 mM Sodium phosphate buffer, pH 7.5, 85 mM Chitobiose, 80 μg enzyme. Reaction with combination of two enzyme was performed in 1 ml of 10 mM Sodium phosphate buffer, pH 7.5 85 mM Chitobiose, 5 mM MgSO₄, 40 mM ATP, 80 μg chitobiose phosphorylase and 70 μg of hexosaminokinase. Reaction mix incubated at 30° C. Product formation was analyzed daily by HPLC.

Product Analysis.

Products obtained were analyzed by TLC and HPLC. Thin-layer chromotography (TLC) for product formation was analyzed using Silica gel 60 F254 (EM Science) at mobile phase Butanol/acetic acid/water in a ratio 2:1:1, n-butanol:methanol:25% ammonia solution:water (5:4:2:1 v:v:v:v) or n-butanol:methanol:25% ammonia solution (5:4:3 v:v:v). For staining p-anisaldehyde/acetic acid/H₂SO₄/H2O in ratio 7:3:10:27 was used.

HPLC.

Chitobiose production was analyzed by HPLC using Aminex® HPX-87 column (Bio-Rad) with mobile phase 5 mM H₂SO₄. GlcNAc-1-phosphate was analyzed by HPLC using a Supelcosil LC-SAX column with 0.05 M K-phosphate buffer, pH 4.0. Nucleotide concentrations were analyzed by HPLC using a Supelcosil LC-18-T column with a flow rate of 1.0 mL/min of 0.05 M KH₂PO₄/4 mM tetrabutylammonium hydrogen sulfate, pH 6.0 and a linear gradient solvent program of 0-30% methanol or using a Inertsil ODS-4 (GI Science, Inc) with 0.1 M KH₂PO₄/8 mM tetrabutylammonium hydrogen sulfate, pH 6.4 and a linear gradient solvent program of 0-30% acetonitrile. Sugar-1-phosphate formation was also analyzed by thin-layer chromotography (TLC) using Silica gel 60 F254 (EMD), Butanol/acetic acid/water in a ratio 2/1/1 was for a mobile phase used. For staining used p-anisaldehyde/acetic acid/H₂SO₄/H2O in ratio 7/3/10/27.

Purification.

N,N′-diacetylchitobiose was purified using Bio-Gel® P-2 media (Bio-Rad), GlcNAc-1-phosphate and UDP-GlcNAc were purified using Dowex 1×8 100-200 mesh and eluted: GlcNAc-1-phosphate with 0.4M Potassium acetate buffer, pH5; UDP-GlcNAc eluted with 0.5M NaCl, 0.02N HCl, pH 1.65. Combined and concentrated fractions after Dowex column were desalted using Bio-Gel® P-2 media. Purity was analyzed by TLC and HPLC using Aminex® HPX-87 column.

### Results

Protein Expression.

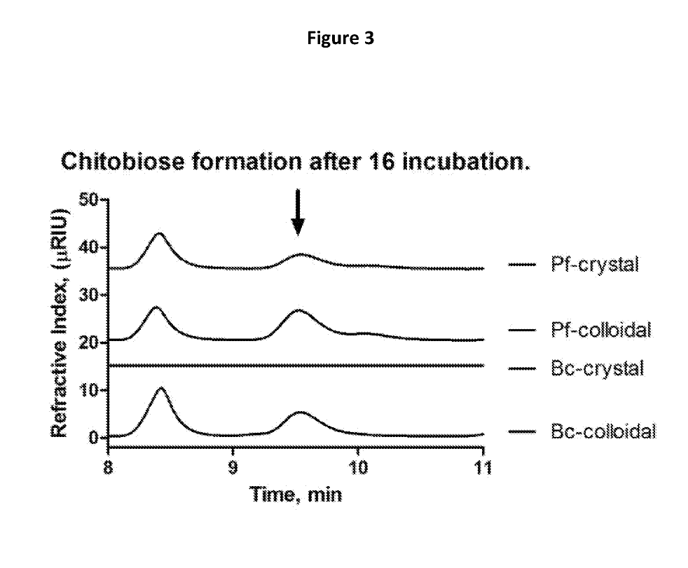

All proteins were cloned into pET-28b vector and expressed in E. coli BL21. All proteins, except P. furiosus chitinase (Chi-Pf), had high level of soluble protein expression (FIG. 3). Expression of Chi-Pf was very low, probably because of a proline-threonine rich linker region. Expression media enrichment with casaminoacid slightly increased expression level, but not significantly.

NAD-dependent epimerase/dehydratase from PC (Pyrobaculum calidifontis JCM 11548), TT (Thermus thermophilus JL18), VM (Vulcanisaeta moutnovskia), EC (Escherichia coli) were cloned into pet15b expression vector, expressed in E. coli and purified using an affinity column using His Tag purification. For protein purification E. coli cells were inoculated into 100 ml of Luria Bertani medium and induced overnight with IPTG at room temperature. Cells were then stored at −20° C. Cells were resuspended in 50 mM NaPi (pH 7.8) buffer and disrupted by using a French press. After centrifugation at 13,000 g, the supernatant was loaded onto 3 ml Cobalt resin (Talon, Clonetech). Protein was purified according manufacturer's protocol, eluted with 50 mM NaPi buffer (pH 7.8) with 300 mM NaCl and 250 mM imidazole. Fractions that contained protein were concentrated using an Amicon 10K ultrafiltration system. All strains of E. coli were grown at 30° C. in 5 mL starter cultures overnight. Next day, 25 mL rich media was inoculated with starter cultures and grown over night at 37° C. Cells were collected by spinning at 5000 rpm at 4° C. for 10 minutes. Cells were washed twice in 50 mL 50 mM NaPi (pH 7.8). Cells were resuspended in 10 mL 50 mM NaPi (pH 7.8). Cultures were placed on ice. Cells were lysed by French press at 20000 psi. Cell lysates were spun at 12000 rpm at 4° C. for 15 minutes. Supernatants were passed through cobalt column twice, and then washed with 10 mM imidazole buffer. All enzymes were eluted with 250 mM imidazole buffer. 1 mL fractions were collected. All fractions that tested positive for protein using Bradford assay were collected and made into 10% glycerol. Protein concentrations were determined by Bradford assay. Using the standard curve, the following concentrations were determined for each purified enzyme.

| Sample | Mass (ug) | Concentration (ug/uL) |
|---|---|---|
| TT | 2.45 | 0.49 |
| VM | 4.24 | 0.85 |
| PC | 0.99 | 0.05 |
| EC | 2.86 | 0.29 |
| EC DESALT | 3.99 | 15.94 |

Samples were run on SDS-PAGE to determine purity.

| Lane: | 1 | 2 | 3 | 4 | 5 | 6 |
|---|---|---|---|---|---|---|
| Sample | Ladder | TP | VM | PC | EC | EC |
| | | | | | | desalted |
| Volume | 5 uL | 20 uL | 20 ul | 20 uL | 20 uL | 10 uL |
| Loaded | | 1:1 | 1:1 | 1:1 | 1:1 | 1:10 |

The epimerase reaction conditions included 100 mM UDP-GlcNAc (50 μl), protein (20 μl), 50 mM NaPi (130 μl), incubate at 37° C., 50° C. and 80° C. for 16 hours. HPLC was performed after 16 hours. Enzyme was stored at 4° C. after purification or at −80° C. with 10% glycerol and dialyzed before reactions. Optimal reactions were found at 37° C.

N,N′-Diacetylchitobiose Preparation.

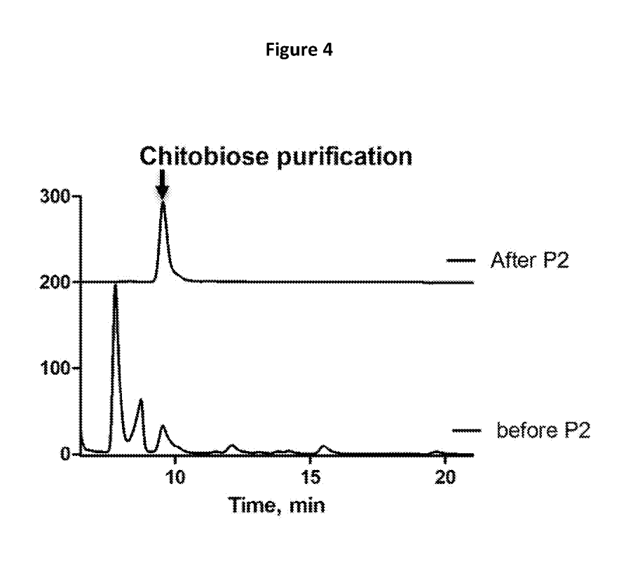

A chitobiose reaction was carried out in small (0.5 ml) and large (0.5 L) scale. For the small-scale reaction, the concentrations of crystal chitin used was 25 mg/ml and colloidal chitin used was 5 mg/ml. The reaction mix was incubated overnight, for P. furiosus chitinase at 95° C. and for B. cereus chitinase at 55° C. Chitobiose formation was detected by HPLC in all reactions, except when B. cereus chitinase was incubated with crystal chitin. After overnight incubation, the reaction with crystal chitin and P. furiosus chitinase was detected and contained 0.4 mg/ml of chitobiose. The reaction with colloidal chitin and either chitinase yielded 0.9 mg/ml chitobiose (FIG. 4).

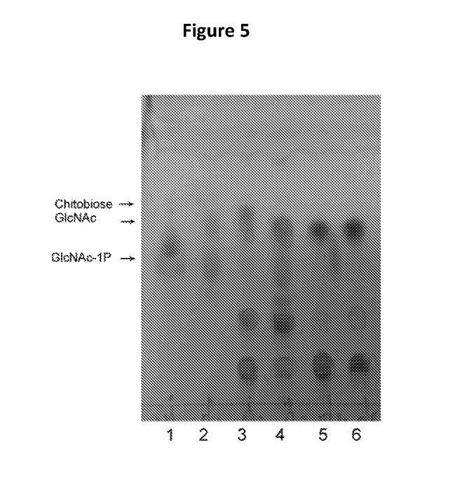

A large-scale reaction was carried out in 0.5 ml volume with 10 g of colloidal chitin (10 g of crystal chitin was used for preparation of colloidal chitin). After 7 days incubation with B. cerius chitinase at 55° C., remaining solid material was separated by centrifugation and concentrated supernatant analyzed by HPLC. The total yield on large scale reaction was approximately 3 g of chitobiose. Chitobiose was purified using Bio-Gel® P-2 media and used for GlucNAc-1-phosphate production (FIG. 5).

### GlcNAc-1-Phosphate Preparation.

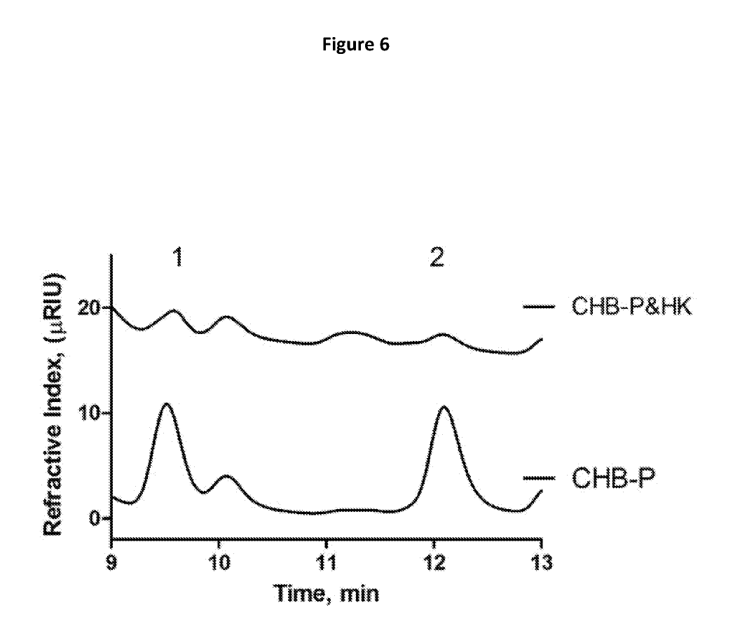

N,N′-diacetylchitobiose phosphorylase from 2 different microorganisms were analyzed (FIG. 6). This included the extreme thermophile Dictyoglomus thermophilum H-6-12. It was expected that its enzyme would be thermostable and thermophilic but it had not been analyzed before. Because the optimum growth temperature for this organism is 50-80° C., enzyme activity was analyzed in a range from 30-80° C. Unfortunately, no activity with either the commercial chitobiose or that prepared in-house was detected. The enzyme from Vibrio proteoliticus had previously demonstrated activity with chitobiose at an optimal temperature of 30° C. This enzyme worked well with both commercial and in-house prepared chitobiose. The phosphorylase reaction is reversible, and it has been shown that the synthetic reaction is inhibited by high concentrations of GlcNAc. Several enzymatic reactions with different substrate and enzyme combinations were performed to attempt to drive the reaction forward: 1) adding extra GlcNAc, with production of GlcNAc-1-phosphate 2) coupling it with the nucleotidyltransferase reaction in order to shift the reaction to completion, with the final product, UDP-GlcNAc, 3) coupling reaction with hexosamine kinase (H K).

The first two reactions did not improve the yield of the reaction. Only in combination with HK did the reaction shift toward the formation of GlcNAc-1-phosphate. Surprisingly, the HK reaction using GlcNAc as a substrate was extremely slow and produced very small amounts of GlcNAc-1-phosphate (FIG. 5, lane 5 and 6).

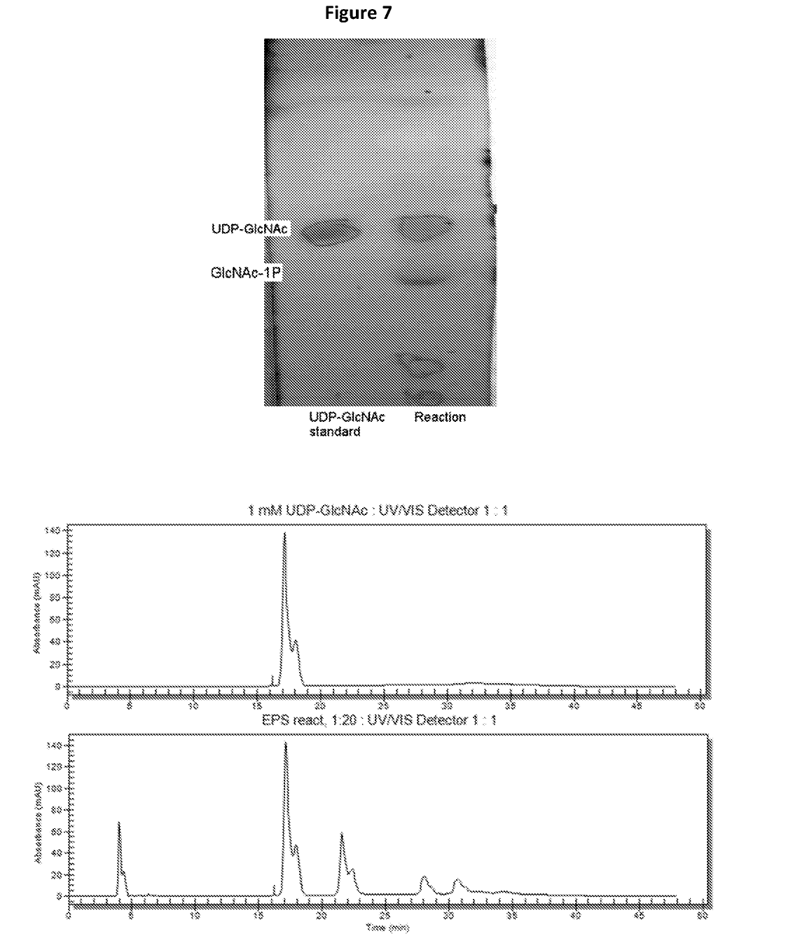

When the two enzymes were used simultaneously, HPLC analysis of the reaction indicated almost complete degradation of chitobiose and an absence of GlcNAc. When only chitobiose phosphorylase was used, the starting substrate was detected in the final product together with a significant amount of GlcNAc-1-phosphate (FIG. 7).

Based on this data, 600 mg of purified chitobiose was used for preparation of GlcNAc-1-phosphate at 10 mL scale. The reaction mix contained 150 mM sodium phosphate buffer, 150 mM of chitobiose, 50 mM MgSO₄, 70 mM ATP, 3.0 mg of CHB-P and 1.7 mg of HK. The reaction was monitored by TLC over 6 days and analyzed by HPLC (FIG. 6, shows depletion of chitobiose and GlcNAc). GlcNAc-1-phosphate was purified first with Dowex 1×8 100-200 mesh resin followed by a P2 bio-gel. Purified GlcNAc-1-phosphate was analyzed using a Supelcosil LC-SAX column.

### 1. Demonstrating Production of UDP-GlcNAc.

Purified GlcNAc-1-phosphate was used for the nucleotidyltransferase reaction (0.5 mL scale, 30 mM of GlcNAc-1-phosphate). The reaction was monitored by TLC and HPLC (lnertsil column). According to the HPLC, approximately 20 mM of UDP-GlcNAc was produced in this reaction (FIG. 7). Reaction conditions will be further optimized in the Phase II work to achieve a maximum conversion rate.

2. Coupling UDP-GlcNAc Production Reaction with UDP-GlcNAc Epimerases for Production of UDP-GalNAc.

Epimerases from E. coli, Vulcanisaeta moutnovskia (GalE-VM) and Thermus thermophilus (GalE-TT). All enzymes were expressed in E. coli at high levels of soluble protein and were able to convert UDP-GlcNAc to UDP-GalNAc at approximately the same rate with about 10-25% yield (based on HPLC analysis). E. coli GalE had activity at 30-37° C., while the GalE-TT and GalE-VM were active within a broad range of temperatures (37° C.-70° C.). Using enzymes from thermophilus had an advantage because they allowed us to skip an extensive protein purification step. In addition, enzymes from thermophilus were more stable and have longer storage life.

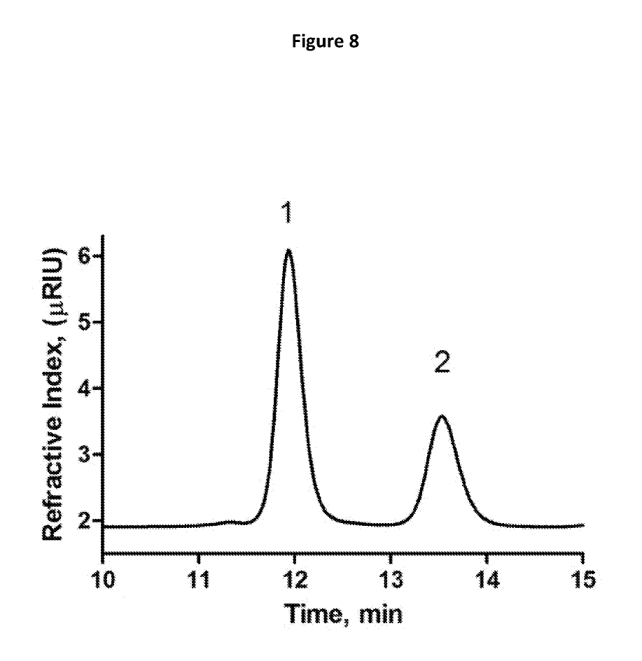

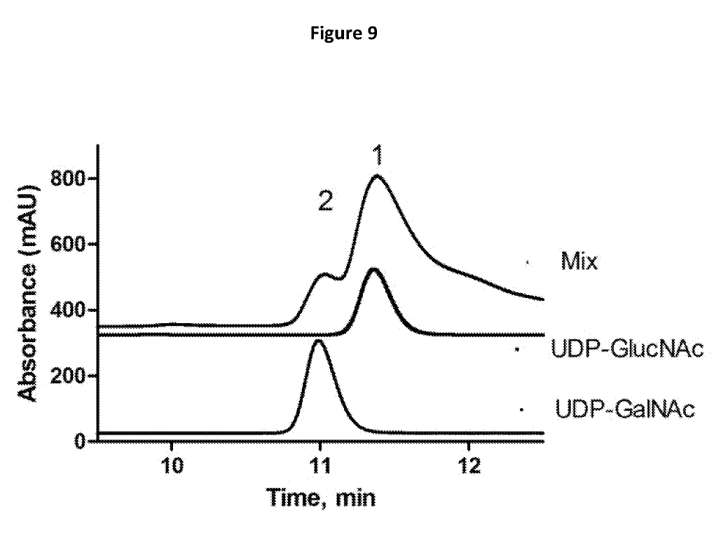

Detection of products in the epimerization reaction were attempted using 2 methodologies: 1) acid degradation of activated sugars and detection with Amenex HPX-87 column and 2) direct detection using Inertsil ODS-4 column. Both methods have their own disadvantage. The acid degradation method gave a good separation of GlcNAc and Gal NAc (FIG. 8), but needed the degradation step which was not complete, making it difficult to estimate conversion rate correctly. The direct detection method did not separate the two activated sugars well. UDP-GalNAc appeared as an extra peak on the left arm of UDP-GlcNAc peak (FIG. 9). Nevertheless we were able to demonstrate coupling of the reaction.

3. Coupling of UDP-GlcNAc/GalNAc Production Reaction with Glycosyltransferase Reaction for Production of Lacto-N-Triose II and Globotetraose.

To produce lacto-N-triose II and globotetraose we used two glycosyltransferases from N. gonorrhoeae: LgtA and LgtD. Both enzymes are known to be very specific for their sugar donor. Our plan was to use these enzymes in order to remove one of these activated sugars from the reaction mix and produce only the desired oligosaccharides. LgtA attaches GlcNAc to lactose and produces the important milk oligosaccharide precursor, lacto-N-triose II (GlcNAc-(β1-3)-Gal-(β1-4)-Glc). LgtD attaches GalNAc to globotriose to produce globotetraose. Globotriose (produced in-house) was used as acceptor.

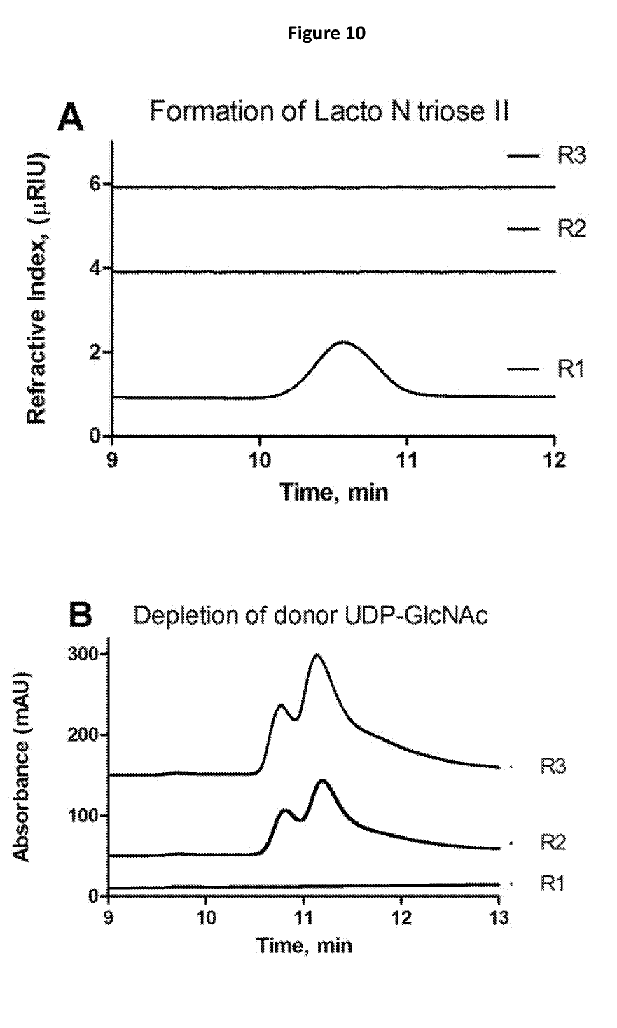

We carried out several sets of reactions. First using pure, commercially available donors to confirm enzyme activity, both LgtA (R1 in FIG. 10) and LgtD (not shown) had activity. We also successfully demonstrated the use of LgtA using UDP-GlcNAc produced in-house. Second, we analyzed the activity of LgtA in mixture of commercially available donors (FIG. 10, R2). Third, we coupled the epimerization reaction (after epimerase inactivation) using LgtA to remove UDP-GlcNAc from the reaction mix (FIG. 10, R3). Fourth, we tried to increase globotetraose production by coupling the epimerase reaction with LgtD (data not shown). The activity of the enzymes were determined both with product formation (FIG. 10, A) and activated sugar level decrease in reaction mix (FIG. 10 B).

We observed in these experiments that product formation occurred only in the reaction with one donor, but not with a mixture of UDP-GalNAc and UDP-GlcNAc (FIG. 10, A). We also did not detect a decrease of UDP-GlcNAc during glycosyltransferase reaction when using a mixture of donors. This clearly indicates enzyme inhibition by non-specific donor.

### Summary

In this work we have demonstrated feasibility of using our approach to produce UDP-GlcNAc and UDP-GalNAc from chitin by cloning and expressing a variety of enzymes as recombinant proteins in E. coli to degrade chitin to N,N-diacetylchitobiose and produce GlcNAc-1-P, UDP-GlcNAc, UDP-GalNAc, lacto-N-triose II and globotetraose. We have also successfully demonstrated the use of UDP-GlcNAc and UDP-Gal-NAc in glycosyltransferase reactions.

We claim:

1. A method of synthesizing uridine-5′diphospho-N-acetylglucosamine (UDP-GlcNAc) comprising: (a) contacting chitin with an exochitanase to produce N,N-diacetylchitobiose, (b) contacting N,N-diacetylchitobiose with a N-N-diacetylchitobiose phosphorylase to produce GlcNAc-1-phosphate, and (c) contacting GlcNAc-1-phosphate with an uridylyltransferase to produce UDP-GlcNAc.

2. The method of claim 1, wherein the step of contacting N,N-diacetylchitobiose with a N-N-diacetylchitobiose phosphorylase produces GlcNAc.

3. The method of claim 2, wherein the GlcNAc is contacted with a N-acetylhexosamine kinase to produce GlcNAc-1-phosphate.

4. A method of synthesizing uridine-5′diphospho-N-acetylgalactosamine (UDP-GalNAc) comprising: (a) contacting chitin with an exochitanase to produce N,N-diacetylchitobiose, (b) contacting N,N-diacetylchitobiose with a N-N-diacetylchitobiose phosphorylase to produce GlcNAc-1-phosphate, (c) contacting GlcNAc-1-phosphate with an uridylyltransferase to produce UDP-GlcNAc, and (d) contacting UDP-GlcNAc with a UDP-glucose-4-epimerase to produce UDP-GalNAc.

5. The method of claim 4, wherein the step of contacting N,N-diacetylchitobiose with a N-N-diacetylchitobiose phosphorylase produces GlcNAc.

6. The method of claim 5, wherein the GlcNAc is contacted with a N-acetylhexosamine kinase to produce GlcNAc-1-phosphate.

7. A method of synthesizing uridine-5′diphospho-N-acetylglucosamine (UDP-GlcNAc) comprising: (a) contacting N,N-diacetylchitobiose with a N-N-diacetylchitobiose phosphorylase to produce GlcNAc-1-phosphate, and (b) contacting GlcNAc-1-phosphate with an uridylyltransferase to produce UDP-GlcNAc.

8. The method of claim 7, wherein the step of contacting N,N-diacetylchitobiose with a N-N-diacetylchitobiose phosphorylase produces GlcNAc.

9. The method of claim 8, wherein the GlcNAc is contacted with a N-acetylhexosamine kinase to produce GlcNAc-1-phosphate.

10. A method of synthesizing uridine-5′diphospho-N-acetylgalactosamine (UDP-GalNAc) comprising: (a) contacting N,N-diacetylchitobiose with a N-N-diacetylchitobiose phosphorylase to produce GlcNAc-1-phosphate, (b) contacting GlcNAc-1-phosphate with an uridylyltransferase to produce UDP-GlcNAc, and (c) contacting UDP-GlcNAc with a UDP-glucose-4-epimerase to produce UDP-GalNAc.

11. The method of claim 10, wherein the step of contacting N,N-diacetylchitobiose with a N-N-diacetylchitobiose phosphorylase produces GlcNAc.

12. The method of claim 11, wherein the GlcNAc is contacted with a N-acetylhexosamine kinase to produce GlcNAc-1-phosphate.

13. A method of synthesizing uridine-5′diphospho-N-acetylglucosamine (UDP-GlcNAc) comprising: (a) contacting GlcNAc with N-acetylhexosamine to produce GlcNAc-1-phosphate, and (b) contacting GlcNAc-1-phosphate with an uridylyltransferase to produce UDP-GlcNAc.

14. A method of synthesizing uridine-5′diphospho-N-acetylgalactosamine (UDP-GalNAc) comprising: (a) contacting GlcNAc with N-acetylhexosamine to produce GlcNAc-1-phosphate, (b) contacting GlcNAc-1-phosphate with an uridylyltransferase to produce UDP-GlcNAc, and (c) contacting UDP-GlcNAc with a UDP-glucose-4-epimerase to produce UDP-GalNAc.

15. The method of claim 1, 4, 7, 10, 13, or 14, wherein the reactions are carried out with sequential addition of enzymes.

16. The method of claim 1, 4, 7, 10, 13, or 14, wherein the reactions are carried out with a simultaneous addition of enzymes.

17. The method of claim 1, 4, 7, 10, 13, or 14, wherein the reactions are carried out in a single reaction vessel.

18. The method of claim 4, 10 or 14, wherein the UDP-glucose-4-epimerase is a GalE-VM epimerase (SEQ ID NO:2) or GalE-TT epimerase (SEQ ID NO:4) or GalE-PC epimerase (SEQ ID NO:6).

19. A method of synthesizing lacto-N-triose II, comprising: contacting lactose and UDP-GlcNAc with a glycosyltransferase to produce lacto-N-triose II.

20. A method of synthesizing globotetraose, comprising: contacting globotriose and UDP-GlcNAc with a glycosyltransferase to produce globotetraose.


SEQUENCE LISTING (6 sequences)

SEQ ID NO: 1 - DNA, 933 bp, Vulcanisaeta moutnovskia
atgggcgagacaatactaatcactggcggggctgggttcataggtagccacatggtggataggttagtcaacgagggcttcaaggtcagggtaatcgacaacctaagtagcggcaggctggaaaacctgaagcacctcggcacctcaatagaggccatgattggtgacttgaagaaaccagaggacgcactcaaggcagtagacggggtggatgcagtcttccacttcgctgcgaatccggaggttagggtaagcaccacaaacccggagacccacttcaacgagaatgtggtggccacattcaacctacttgaggcaatgaggaggagaggcgttaaggagttggtcttcgcgtcctcaagctcggtctacggcgaaccaagcgaaataccagtcggtgaggacgcgccgattaggccggtctcggtgtacggagcaagcaaggcggcttgcgaaaacctaatacacgcctacacgagactctacggcattagggccgtggtgctcaggtacgccaatgtcgttggaccaaggctaagacacggcgtcgtctgggacttcatgaacaagctcaggactaacccaagggagcttgaggtgcttggcaacggtacccaggtcaggagttacatatacattgatgatgccgtggaggcgacaatactggcctggagaaaggctagtgatgcattcaatgtctttaacgtcgcgtcagaggattggataaccgtagacgaggtggctaagatagtcatcgagaccatggggctcaaggacgtgaaaataatccataaaccagtcgcgcatggagttggctggctaggtgacgtaaagaaaatagcacttaaaatagagaggttaaaggcattaggcttcaaaccgaaattaaatagcgcacaggctgttaaggaaaccgttctgggcatgatgtag

SEQ ID NO: 2 - protein, 310 aa, Vulcanisaeta moutnovskia
MGETILITGGAGFIGSHMVDRLVNEGFKVRVIDNLSSGRLENLKHLGTSIEAMIGDLKKPEDALKAVDGVDAVFHFAANPEVRVSTTNPETHFNENVVATFNLLEAMRRRGVKELVFASSSSVYGEPSEIPVGEDAPIRPVSVYGASKAACENLIHAYTRLYGIRAVVLRYANVVGPRLRHGVVWDFMNKLRTNPRELEVLGNGTQVRSYIYIDDAVEATILAWRKASDAFNVFNVASEDWITVDEVAKIVIETMGLKDVKIIHKPVAHGVGWLGDVKKIALKIERLKALGFKPKLNSAQAVKETVLGMM

SEQ ID NO: 3 - DNA, 936 bp, Thermus thermophiles
atgcgcgtgctggtgacgggcggcgccggtttcatcggaagccacatcgtggaggacctcctcgccaggggcctggaggtggcggtcctggacaacctggccacgggcaagcgggaaaacgtgcccaaaggcgtccccttcttccaggtggacctaagggacaaggaggaggtggagcgggccttccgggagttccgccccacccacgtctcccaccaggcggcccaggcctcggtgaaggtgagcgtggaggacccggtcctggactttgaggtgaacctcctgggggggctcaacctcctcgaggcctgccgccagtacggcgtggagaagcttgtcttcgcctccacgggcggggccatctacggggaggtgcccgagggggaaagggccgaggagacctggcccccccgcccaaaaagcccctacgccgcgagcaaggcggcctttgagcactacctctccgtctacgggcaaagctacgggctcaagtgggtctccctgcggtacggcaacgtgtacggccccaggcaggacccccacggggaggcgggggtggtggccatcttcgccgagcgggtcctcaatggcctgcccgtgaccctctacgcccggaagacccccggggacgaggggtgcgtgcgggactacgtctacgtgggggacgtggccgaggcccacgccctcgccctcttctccctggaagggatctacaacgtggggaccggggaggggcacaccacgagggaggtcctcgaggcggtggccgaggcggcggggaaggccccccaggtccagcccgccccgccgcgccccggggacctggaaaggagcgtcctctcccccctgaagctcatggcccacggctggcggcccaaggtggggttccaggagggcatccgcctcaccgtggaccacttccggggcgccgtatag

SEQ ID NO: 4 - protein, 311 aa, Thermus thermophiles
MRVLVTGGAGFIGSHIVEDLLARGLEVAVLDNLATGKRENVPKGVPFFQVDLRDKEGVERAFREFRPTHVSHQAAQASVKVSVEDPVLDFEVNLLGGLNLLEACRQYGVEKLVFASTGGAIYGEVPEGERAEETWPPRPKSPYAASKAAFEHYLSVYGQSYGLKWVSLRYGNVYGPRQDPHGEAGVVAIFAERVLKGLPVTLYARKTPGDEGCVRDYVYVGDVAEAHALALFSLEGIYNVGTGEGHTTREVLEAVAEAAGKAPLVQPAPPRPGDLERSVLSPLKLMAHGWRPKVGFQEGIRLTVDHFRGAV

SEQ ID NO: 5 - DNA, 939 bp, Pyrobaculum calidifontis
atgagaattgttgtaacaggcggcgctgggttcattggtagccacctcgtggacaagctcgtggagcttgggtacgaggtcgtcgtcgtggacaacctctcgagcggcagacgcgagtttgtcaaccccagcgcagagctccacgtcagagacctcaaggactactcctggggcgcggggatcaaaggcgacgtagttttccacttcgccgcaaaccccgaagtccgcctatccaccacagagcccatcgttcacttcaacgagaacgtggtagccactttcaacgtgttagagtgggcgaggcagacgggggtgaggactgtggtatttgcctcatcctccaccgtgtacggcgacgccgacgtcattccaacgccggaggaagagccgtacaagccgatatcggtctacggcgcggcaaaggcggcgggcgaggtcatgtgcgccacctacgccaggctattcggcgtccggtgcctcgcggttaggtacgcgaatgtggtagggccccggcttaggcacggcgtaatatacgactttataatgaagctcaggagaaaccccaacgtcctcgaggtgctgggagacgggacgcagagaaagagctacctctacgtcagagacgccgtagaggccacgttggccgcgtggaagaagttcgaggagatggatgcccccttcctagccctcaacgtgggcaacgtagacgcggtgagggtgttagacattgcgcagatagtggcagaggtcttggggctaaggcccgagattaggctagtcccctctaccccagacgggagggggtggcccggagacgtgaagtacatgacactcgcagtgacaaagctcatgaagctgacggggtggaggcccactatgacaagcgccgaggccgttaaaaagacggcggaggacctggcaaaagagctatggggctag

SEQ ID NO: 6 - protein, 312 aa, Pyrobaculum calidifontis
MRIVVTGGAGFIGSHLVDKLVELGYEVVVVDNLSSGRREFVNPSAELHVRDLKDYSWGAGIKGDVVFHFAANPEVRLSTTEPIVHFNENVVATFNVLEWARQTGVRTVVFASSSTVYGDADVIPTPEEEPYKPISVYGAAKAAGEVMCATYARLFGVRCLAVRYANVVGPRLRHGVIYDFIMKLRRNPNVLEVLGDGTQRKSYLYVRDAVEATLAAWKKFEEMDAPFLALNVGNVDAVRVLDIAQIVAEVLGLRPEIRLVPSTPDGRGWPGDVKYMTLAVTKLMKLTGWRPTMTSAEAVKKTAEDLAKELWG